(19) United States
(12) Patent Application Publication
(10) Pub. No.: US 2020/0375937 A1
(43) Pub. Date: Dec. 3, 2020

(54) MCT-BASED NUTRITION BLEND FOR PROVIDING HEALTH BENEFITS IN ANIMALS

(71) Applicant: Société des Produits Nestlé S.A., Vevey (CH)

(72) Inventor: Yuanlong Pan, Chesterfield, MO (US)

(21) Appl. No.: 16/886,955

(22) Filed: May 29, 2020

Related U.S. Application Data
(60) Provisional application No. 62/855,045, filed on May 31, 2019.

(51) Int. Cl. A61K 31/232; A61K 31/231; A61K 9/00; A61K 31/198; A61K 31/375; A61K 31/355; A61P 3/02

(52) U.S. Cl. CPC A61K 31/232; A61K 31/231; A61K 9/0056; A61P 3/02; A61K 31/375; A61K 31/355; A61K 31/198

(57) ABSTRACT

A method for treating sarcopenia or muscle atrophy in an animal and a method of increasing weight gain, increasing fat gain, or maintaining lean body mass in an animal in need thereof, is provided. The methods can comprise orally administering a composition comprising a therapeutically effective amount of medium chain triglycerides to the animal.

### CROSS REFERENCE TO RELATED APPLICATIONS

This application claims priority to U.S. Provisional Application Ser. No. 62/855,045 filed May 31, 2019, the disclosure of which is incorporated in its entirety herein by this reference.

### BACKGROUND

During aging, there is a gradual decrease in the ability to maintain skeletal muscle function and mass. The condition is known as “sarcopenia.” The exact cause of sarcopenia is unknown, but may be due to a combination of the gradual failure of “satellite cells” that help to regenerate skeletal muscle fibers and a decrease in sensitivity to or the availability of critical secreted growth factors that are necessary to maintain muscle mass and ensure satellite cell survival. Methods for combating sarcopenia are known in the art. U.S. Pat. No. 7,442,706 disclose methods for treating sarcopenia with growth hormone secretagogues. U.S. Pat. Nos. 7,232,580 and 7,138,148 disclose the use of extracts of Ginkgo biloba for preparing a medicament for the treatment of sarcopenia.

Similarly, muscle atrophy is defined as a decrease in the mass of the muscle. Muscle atrophy can be a partial or complete wasting away of muscle. When a muscle atrophies, it becomes weaker and, since the ability to exert force is related to mass, loses its ability to effectively support body functions, e.g., motion. Muscle atrophy can be caused by many factors, e.g., diseases such as cancer, AIDS, congestive heart disease, chronic obstructive pulmonary disease, renal failure, and severe burns. Often these animals suffer from “cachexia” and have a poor prognosis for good health or survival. Methods for combating muscle atrophy are known in the art. US20070122821 discloses antisense compositions and methods for treating muscle atrophy. US20060003959 discloses methods and agents for maintaining muscle mass and for preventing muscle atrophy and related biomarkers.

Despite these known methods, problems attributable to sarcopenia and muscle atrophy persist. There is, therefore, a need for novel methods for combating sarcopenia and muscle atrophy in animals, particularly aging animals.

### SUMMARY

The present disclosure relates to compositions comprising medium-chain triglycerides and methods comprising administering the compositions to an animal to provide a health benefit. More specifically, the present disclosure relates to compositions that comprise medium-chain triglycerides (MCT) and, in some aspects, can optionally include one or more of omega-3 fatty acids, antioxidants, or arginine. The present disclosure also relates to methods for treating sarcopenia or muscle atrophy and to methods of increasing weight gain, increasing fat gain, or maintaining lean body mass in an animal in need thereof by administering the present MCT compositions to the animal.

The present inventors have discovered that the present MCT compositions can treat sarcopenia or muscle atrophy in an animal as well as increase weight gain, increase fat gain, or maintain lean body mass in an animal in need thereof. Such effect can help treat such animals suffering from cancer, AIDS, congestive heart disease, chronic obstructive pulmonary disease, renal failure, severe burns, and cachexia. Accordingly, in a general embodiment, a method of treating sarcopenia or muscle atrophy in an animal as well as increasing weight gain, increasing fat gain, or maintaining lean body mass in an animal in need thereof is provided. The methods can comprise orally administering a composition comprising a therapeutically effective amount of medium chain triglycerides to the animal.

Additional features and advantages are described herein and will be apparent from, the following Detailed Description.

### DETAILED DESCRIPTION

### Definitions

Some definitions are provided hereafter. Nevertheless, definitions may be located in the “Embodiments” section below, and the above header “Definitions” does not mean that such disclosures in the “Embodiments” section are not definitions.

As used in this disclosure and the appended claims, the singular forms “a,” “an” and “the” include plural referents unless the context clearly dictates otherwise. Thus, for example, reference to “an ingredient” or “the ingredient” includes two or more ingredients. The term “and/or” used in the context of “X and/or Y” should be interpreted as “X,” or “Y,” or “X and Y.” Where used herein, the term “example,” particularly when followed by a listing of terms, is merely exemplary and illustrative, and should not be deemed to be exclusive or comprehensive.

As used herein, “about” is understood to refer to numbers in a range of numerals, for example the range of −10% to +10% of the referenced number, preferably within −5% to +5% of the referenced number, more preferably within −1% to +1% of the referenced number, most preferably within −0.1% to +0.1% of the referenced number. A range that is “between” two values includes those two values. Furthermore, all numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

All percentages expressed herein are by weight of the total weight of the composition on a dry matter basis unless expressed otherwise. When reference is made to the pH, values correspond to pH measured at 25° C. with standard equipment.

The terms “food,” “food product” and “food composition” mean a product or composition that is intended for ingestion by an animal and provides at least one nutrient to the animal. The term “pet food” means any food composition intended to be consumed by a companion animal. Such food compositions can include main meal, treats, beverages, supplements, etc.

The term “companion animal” means a dog or a cat. As used herein, the term “cat” and “feline” can be used interchangeably. Additionally, the term “dog” and “canine” can be used interchangeably. In one embodiment, the companion animal can be a cat.

The term “aging” means being of advanced age such that the animal has exceeded 50% of the average lifespan for its particular species and/or breed within a species. For example, if the average lifespan for a given breed of dog is 10 years, then a dog within that breed greater than 5 years old would be considered “aging” for purposes herein. Additionally, for example, if the average lifespan for a breed of cat is 15 years, then a cat within that breed greater than 7.5 years old would be considered “aging” for the purposes herein. In an embodiment, the compositions and methods disclosed herein involve a senior animal, e.g. a senior dog or a senior cat. Animals, such as dogs and cats, are considered senior in the last 25% of their lives. As noted herein, the life span of a dog or a cat depends on its size and/or its breed, and a senior dog or senior cat can be determined based on the above calculation but using the numerical value of 75% such that the age threshold is exceeding 75% of the average lifespan.

“Wet food” means a pet food having a moisture content from about 50% to about 90%, and in one aspect, from about 70% to about 90%. “Dry food” means a pet food having a moisture content less than about 20%, and in one aspect, less than about 15%, and in a specific aspect, less than about 10%. “Semi-moist food” means a pet food having a moisture content from about 20% to about 50%, and in one aspect, from about 25% to about 35%. “Kibbles” means pieces of dry or semi-moist pet food which can have a pellet shape or any other shape. Non-limiting examples of kibbles include particulates; pellets; pieces of pet food, dehydrated meat, meat analog, vegetables, and combinations thereof; and pet snacks, such as meat or vegetable jerky, rawhide, and biscuits.

The compositions disclosed herein may lack any element that is not specifically disclosed herein. Thus, a disclosure of an embodiment using the term “comprising” includes a disclosure of embodiments “consisting essentially of” and “consisting of” the components identified. Similarly, the methods disclosed herein may lack any step that is not specifically disclosed herein. Thus, a disclosure of an embodiment using the term “comprising” includes a disclosure of embodiments “consisting essentially of” and “consisting of” the steps identified. Moreover, the description of some steps as “optional” does not imply that the other steps which are not explicitly described as optional are necessarily required.

Any embodiment disclosed herein can be combined with any other embodiment disclosed herein.

“Prevention” includes reduction of risk and/or severity of a condition or disorder. The terms “treatment,” “treat” and “to alleviate” include both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of patients at risk of contracting a disease or suspected to have contracted a disease, as well as patients who are ill or have been diagnosed as suffering from a disease or medical condition. The term does not necessarily imply that a subject is treated until total recovery. The terms “treatment” and “treat” also refer to the maintenance and/or promotion of health in an individual not suffering from a disease but who may be susceptible to the development of an unhealthy condition. The terms “treatment,” “treat” and “to alleviate” are also intended to include the potentiation or otherwise enhancement of one or more primary prophylactic or therapeutic measure. The terms “treatment,” “treat” and “to alleviate” are further intended to include the dietary management of a disease or condition or the dietary management for prophylaxis or prevention a disease or condition. A treatment can be patient- or doctor-related.

The relative terms “improved,” “increased,” “enhanced” and the like refer to the effects of the composition disclosed herein (a composition comprising a therapeutically effective amount of medium chain triglycerides or a prophylactic dose of medium chain triglycerides) relative to a composition having a lower amount or lacking medium chain triglycerides, but otherwise identical.

The term “therapeutically effective amount” means an amount of a compound of the present invention that (i) treats or prevents the particular disease, condition, or disorder, (ii) attenuates, ameliorates, or eliminates one or more symptoms relating thereto of the particular disease, condition, or disorder, or (iii) prevents or delays the onset of one or more symptoms of the particular disease, condition, or disorder described herein. In one embodiment, therapeutically effect amount can refer to the amount to treat any condition relating to a sarcopenia or muscle atrophy.

The term “long-term administration” means periods of repeated administration or consumption in excess of one month. Periods of longer than two, three, or four can be used for certain embodiments. Also, more extended periods that include longer than 5, 6, 7, 8, 9, or 10 months can be used. Periods in excess of 11 months or 1 year can also be used. Longer term use extending over 1, 2, 3, or more years can also be included in the invention. For certain aging animals, the animal will continue consuming on a regular basis for the remainder of its life. Sometimes this is referred to as consumption for “extended” periods.

The term “regular basis” means at least monthly dosing with the compositions or consumption of the compositions, and in some aspects, weekly dosing. More frequent dosing or consumption, such as twice, three, or seven times weekly, can be used in certain embodiments. Still other embodiments include regimens that comprise at least once daily consumption. The skilled artisan will appreciate that dosing frequency will be a function of the composition that is being consumed or administered, and some compositions may require more or less frequent administration to maintain a desired level of hydration.

A “medium chain triglyceride” is a lipid in which three fatty acids are bound by ester linkages to a glycerol backbone, and at least two and preferably all three of the fatty acids are each between six and twelve carbons in length. The medium-chain fatty acids are caproic acid (comprising six carbon atoms or C6:0), caprylic acid (comprising eight carbon atoms or C8:0), capric acid (comprising ten carbon atoms or C10:0) and lauric acid (comprising twelve carbon atoms or C12:0). In one embodiment, the medium-chain fatty acids are mainly (e.g., at least 98%) in the form of triglycerides. A composition comprising “lipids consisting essentially of medium chain triglycerides” contains medium chain triglycerides as at least 20% of the lipids, in some embodiments at least 30% of the lipids, in other embodiments at least 40% of the lipids, and in some embodiments at least 50% of the lipids in the composition. In one embodiment, the medium chain fatty acids of the medium chain triglycerides can include at least 50% caprylic acid of the total medium chain fatty acids that are present. In another embodiment, the medium chain fatty acids of the medium chain triglycerides can include at least 90% caprylic acid of the total medium chain fatty acids that are present.

### EMBODIMENTS

The present disclosure relates to compositions comprising medium-chain triglycerides and methods comprising administering the compositions to an animal to provide a health benefit. More specifically, the present disclosure relates to compositions that comprise medium-chain triglycerides (MCT) and, in some aspects, can optionally include one or more of omega-3 fatty acids, antioxidants, or arginine. The present disclosure also relates to methods for treating sarcopenia or muscle atrophy and to methods of increasing weight gain, increasing fat gain, or maintaining lean body mass in an animal in need thereof by administering the present MCT compositions to the animal.

The present inventors have discovered that the present MCT compositions can treat sarcopenia or muscle atrophy in an animal as well as increase weight gain, increase fat gain, or maintain lean body mass in an animal in need thereof. Such effect can help treat such animals suffering from cancer, AIDS, congestive heart disease, chronic obstructive pulmonary disease, renal failure, severe burns, and cachexia. Accordingly, in a general embodiment, a method of treating sarcopenia or muscle atrophy in an animal as well as increasing weight gain, increasing fat gain, or maintaining lean body mass in an animal in need thereof is provided. The methods can comprise orally administering a composition comprising a therapeutically effective amount of medium chain triglycerides to the animal.

The methods can further include an identifying step for which treatment is sought or for which a health benefit is sought. For example, the method can further comprise identifying an animal having sarcopenia or muscle atrophy. In other embodiments, the method can further comprise identifying an animal in need of one of the following: increase weight gain, increase fat gain, or maintain lean body mass, and combinations thereof. In still other embodiments, the method can further comprise identifying an animal suffering from cancer, AIDS, congestive heart disease, chronic obstructive pulmonary disease, renal failure, severe burns, and cachexia.

The methods generally comprise orally administering to the animal a composition comprising medium chain triglycerides and optionally one or more of omega-3 fatty acids, antioxidants, and/or an amino acid. In one embodiment, the composition can further comprise a component selected from the group consisting of an omega-3 fatty acid, antioxidants (including vitamin E, vitamin C, selenium, and/or polyphenols), arginine, and mixtures thereof. The composition can be a pet food, such as a wet pet food, a semi-moist pet food, or a dry pet food, e.g., kibble.

Generally, the medium chain triglycerides can be about 0.5 wt % to about 60 wt % of the composition. In one aspect, the medium chain triglycerides can be from about 1 wt % to about 20 wt % of the composition. In other aspects, the medium chain triglycerides can be from about 1 wt % to about 15 wt %, from about 1 wt % to about 10 wt %, or from about 2 wt % to about 10 wt % of the composition. The medium chain triglycerides may be prepared by any known process, such as direct esterification, rearrangement, fractionation and/or transesterification. For example, the medium chain triglycerides may be prepared from a source of vegetable oil, such as coconut oil, through a rearrangement process. The chain length and distribution thereof may vary depending on the source oil. For example, MCTs containing 1-10% C6, 30-60% C8, 30-60% C10 and 1-10% C12 can be derived from palm oil and/or coconut oil; in some embodiments, at least a portion of the MCTs are provided by coconut oil, but in other embodiments the composition does not contain coconut oil. MCTs containing at least about 95% C8 can be made by semi-synthetic esterification of octanoic acid to glycerin; in some embodiments thereof, the remainder of the fatty acids are C6 and C10. Mixtures comprising MCTs with about 50% total C8 and/or about 50% total C10 are also useful herein. In one embodiment, the MCTs can include an MCT selected from the group consisting of caprylic acid, capric acid, and a mixture thereof. In one embodiment, the MCTs can substantially include fatty acids that are caprylic acid (C8). For example, such MCTs can include those having at least 50%, at least 55%, at least 60%, at least 65%, at least 70%, at least 75%, at least 80%, at least 85%, at least 95%, at least 98%, or even 100%, caprylic acid.

Non-limiting examples of suitable omega-3 fatty acids include eicosapentaenoic acid (EPA), docosahexaenoic acid (DHA), alpha-linolenic acid (ALA), and mixtures thereof. In one embodiment, the omega-3 fatty acids can range from about 0.2 wt % to about 3 wt % of the composition. In some embodiments, the omega-3 fatty acids are at least about 0.2 wt %, at least about 1.0 wt %, or at least about 2.0 wt %. In one embodiment, the omega-3 fatty acid can be selected from the group consisting of eicosapentaenoic acid (EPA), docosahexaenoic acid (DHA), alpha-linolenic acid (ALA), and mixtures thereof.

In some embodiments, the composition can be administered to the companion animal for a time period of at least one week, at least one month, at least two, three, four, five or six months; and in some embodiments, for at least one year. During the time period, the composition can be administered to the dog at least one day per week, at least two days per week, at least three, four, five or six days per week; or even seven days per week. The composition can be administered in a single dose per day or in multiple separate doses per day. In an embodiment, the composition can be administered in an amount that provides about 0.001 g to 50 g of the MCTs per kg body weight of the companion animal per day. In one aspect, 0.1 g to about 5 g of the MCTs per kg body weight of the companion animal can be administered per day.

In one specific embodiment, the animal can be a companion animal. In one aspect, the companion animal can be a cat. In one embodiment, the animal can be a senior animal or an aging animal. In one aspect, the animal can be a senior cat. In another aspect, the animal can be an aging cat.

In one embodiment, the compositions described herein can be pet food compositions. The pet food compositions disclosed herein can be any food formulated for consumption by a pet such as a cat. In an embodiment, the pet food composition provides complete nutrition as defined by the Association of American Feed Control Officials (AAFCO) and which depends on the type of animal for which the composition is intended (e.g., a cat). In another embodiment, the composition can be a supplement. Such a supplement can be added to a food composition or be administered in conjunction with a food composition, or administered separately. As such, in some embodiments, the present compositions can be complete and nutritionally balanced pet foods.

Generally, pet food composition can comprise protein, carbohydrates, fat, and ash. In various embodiments, the pet food compositions comprise from about 15% to about 50% crude protein. In some embodiments, such compositions may further comprise from about 5% to about 40% fat. In other embodiments, the compositions may further comprise from about 15% to about 60% carbohydrate. In other embodiments, the composition may further comprise from about 0.1% to about 15% ash.

The pet food composition can comprise meat, such as emulsified meat. Examples of suitable meat include poultry, beef, pork, lamb and fish, especially those types of meats suitable for pets. The meat can include any additional parts of an animal including offal. Some or all of the meat can be provided as one or more meat meals, namely meat that has been dried and ground to form substantially uniform-sized particles and as defined by AAFCO. Additionally or alternatively, vegetable protein can be used, such as pea protein, corn protein (e.g., ground corn or corn gluten), wheat protein (e.g., ground wheat or wheat gluten), soy protein (e.g., soybean meal, soy concentrate, or soy isolate), rice protein (e.g., ground rice or rice gluten) and the like.

The pet food compositions disclosed herein can comprise one or more of a vegetable oil, a flavorant, a colorant or water. Non-limiting examples of suitable vegetable oils include soybean oil, corn oil, cottonseed oil, sunflower oil, canola oil, peanut oil, safflower oil and the like. In some embodiments, the lipids in the composition can consist of the MCTs and one or more of any vegetable oil, any fish oil, the lipid from any meat, and any omega-3 fatty acids.

Non-limiting examples of suitable flavorants include yeast, tallow, rendered animal meals (e.g., poultry, beef, lamb, pork), flavor extracts or blends (e.g., grilled beef), animal digests, and the like. Non-limiting examples of suitable colorants include FD&C colors, such as blue no. 1, blue no. 2, green no. 3, red no. 3, red no. 40, yellow no. 5, yellow no. 6, and the like; natural colors, such as caramel coloring, annatto, chlorophyllin, cochineal, betanin, turmeric, saffron, paprika, lycopene, elderberry juice, pandan, butterfly pea and the like; titanium dioxide; and any suitable food colorant known to the skilled artisan.

The pet food compositions disclosed herein can optionally include additional ingredients, such as starches, humectants, oral care ingredients, preservatives, amino acids, fibers, prebiotics, sugars, animal oils, aromas, other oils additionally or alternatively to vegetable oil, salts, vitamins, minerals, probiotic microorganisms, bioactive molecules or combinations thereof.

Non-limiting examples of suitable starches include a grain such as corn, rice, wheat, barley, oats, potatoes, peas, beans, cassava, and the like, and mixtures of these grains, and can be included at least partially in any flour. Non-limiting examples of suitable humectants include salt, sugars, propylene glycol and polyhydric glycols such as glycerin and sorbitol, and the like. Non-limiting examples of suitable oral care ingredients include alfalfa nutrient concentrate containing chlorophyll, sodium bicarbonate, phosphates (e.g., tricalcium phosphate, acid pyrophosphates, tetrasodium pyrophosphate, metaphosphates, and orthophosphates), peppermint, cloves, parsley, ginger and the like. Non-limiting examples of suitable preservatives include potassium sorbate, sorbic acid, sodium methyl para-hydroxybenzoate, calcium propionate, propionic acid, and combinations thereof.

Specific amounts for each additional ingredient in the pet food compositions disclosed herein will depend on a variety of factors such as the ingredient included in the first edible material and any second edible material; the species of animal; the animal's age, body weight, general health, sex, and diet; the animal's consumption rate; the purpose for which the food product is administered to the animal; and the like. Therefore, the components and their amounts may vary widely.

Yet another aspect of the present disclosure is a method of making a pet food, the method comprising adding MCTs to at least one other comestible ingredient, the MCTs are added in an amount effective to provide the health benefits as disclosed herein. For example, the MCTs can be added such that a single serving of the pet food comprises an amount of the MCTs effective to treat or prevent sarcopenia or muscle atrophy.

### Example

By way of example and not limitation, the following non-limiting study is illustrative of compositions and methods using MCTs for controlling or lowering blood pressure or treating kidney or renal disease in a companion animal, in one or more embodiments provided by the present disclosure.

### Example 1—Aging Cat Study

Thirty-two cats were used in this study. The cats ranged in age from 7.6 to 14.4 years at study initiation and there were 13 males and 17 females. The cats were randomized into two groups, and fed either control or the test diet to maintain their body weight during the three-month feeding study. QMR was used to determine their lean body mass, and body fat at the baseline and end of the study. Body weight was recorded at the baseline and end of the study. The diets are shown in table 1.

TABLE 1

| Ingredients | Control | Test diet |
|---|---|---|
| Crude Protein (%) | 41.71 | 40.85 |
| Crude Fat (%) | 17.15 | 21.5 (16% |
| | | LCT + 5.5% |
| | | MCT) |
| Moisture (%) | 7.82 | 6.62 |
| Crude Fiber (%) | 0.66 | 0.69 |
| Ash (%) | 5.56 | 6.01 |
| MCT (%) | 0 | 5.5 |
| DHA (%) | 0 | 0.23 |
| EPA (%) | 0 | 0.33 |
| Arginine (%) | 1.36 | 2.25 |
| Vitamin E (mg/kg) | 81.00 | 823.00 |
| Vitamin C (mg/kg) | 0 | 172 |

Food intake between the two groups is provided in Table 2.

TABLE 2

| Group | Mean (grams) |
|---|---|
| Test | 46.25313 |
| Control | 49.88644 |

As shown in Table 2, the average of food for the control group was higher than the test group. With caloric density of 5.05 kcal/g for the control group and 5.33 kcal/g for the test group, the caloric intake for the control group was slightly higher than the test group (252 kcal for control vs. 247 kcal for test). However, surprisingly, the test group did not lose lean body mass compared to the control group, shown in Table 3. Also, unexpectedly, the test group actually increased body fat as compared to the control group, see Table 4, and body weight, see Table 5.

TABLE 3

| Group | Baseline (Mean kg) | Treatment (Mean kg) |
|---|---|---|
| Test | 3.72 | 3.72 |
| Controls | 4.02 | 3.81 |

TABLE 4

| Code | PHASE | Mean % body Fat |
|---|---|---|
| Test | Baseline | 26.43 |
| Test | End of Study | 29.09 |
| Control | Baseline | 25.45 |
| Control | End of Study | 26.86 |

TABLE 5

| | Change in body weight from |
|---|---|
| Diet | baseline (g) |
| Control | 0 |
| Test | 233 |

As discussed above, the results showed that the cats fed the test diet were able to gain more % body fat than the cats fed the control diet (2.66% vs 1.41%). The test diet increased body fat in senior cats more than a control diet with identical levels of dietary protein and carbohydrate, and lower level of LCT fat (16% vs 17.15%). This finding is unexpected especially because MCTs and omega-3 PUFAs are known for their weight loss benefits. Additionally, the results showed that the cats fed the test diet were able to gain body weight while the cats fed the control diet did not gain any body weight. This observation is opposite to the weight loss benefits of omega-3 PUFAs and MCTs in other species.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A method for treating sarcopenia or muscle atrophy in an animal, the method comprising orally administering a composition comprising a therapeutically effective amount of medium chain triglycerides to the animal.

2. The method of claim 1, wherein the medium chain triglycerides are about 0.5 wt % to about 60 wt % of the composition.

3. The method of claim 1, wherein the medium chain triglycerides are about 1 wt % to about 15 wt % of the composition.

4. The method of claim 1, wherein the medium chain triglycerides include a medium-chain fatty acid selected from the group consisting of caprylic acid, capric acid, and a mixture thereof.

5. The method of claim 1, wherein the composition further comprises a component selected from the group consisting of an omega-3 fatty acid, antioxidants, arginine, and mixtures thereof.

6. The method of claim 5, wherein the omega-3 fatty acid is selected from the group consisting of eicosapentaenoic acid (EPA), docosahexaenoic acid (DHA), alpha-linolenic acid (ALA), and mixtures thereof.

7. The method of claim 1, wherein the composition is a complete and nutritionally balanced pet food.

8. The method of claim 1, wherein the animal is a companion animal.

9. The method of claim 1, wherein the animal is a cat.

10. The method of claim 1, wherein the animal is a senior animal.

11. The method of claim 1, wherein the composition is administered to the animal daily for at least one week.

12. The method of claim 1, wherein the composition is administered in an amount that provides about 0.001 g to about 50.0 g of the MCTs per kg body weight of the animal per day.

13. A method of increasing weight gain, increasing fat gain, or maintaining lean body mass in an animal in need thereof, the method comprising orally administering a composition comprising a therapeutically effective amount of medium chain triglycerides to the animal.

14. The method of claim 13, wherein the medium chain triglycerides are about 1 wt % to about 15 wt % of the composition.

15. The method of claim 13, wherein the composition further comprises a component selected from the group consisting of an omega-3 fatty acid, antioxidants, arginine, and mixtures thereof.

16. The method of claim 13, wherein the composition is a complete and nutritionally balanced pet food.

17. The method of claim 13, wherein the animal is a companion animal.

18. The method of claim 13, wherein the animal is a senior cat.

19. The method of claim 13, wherein the animals in need are from post-surgery recovery, or suffering from injuries, cancer, AIDS, congestive heart disease, chronic obstructive pulmonary disease, renal failure, severe burns, or cachexia.

20. The method of claim 13, wherein the composition is administered in an amount that provides about 0.001 g to about 50.0 g of the MCTs per kg body weight of the animal per day.
